(19) United States
(12) Patent Application Publication
(10) Pub. No.: US 2016/0263050 A1
(43) Pub. Date: Sep. 15, 2016

(54) METHOD FOR INDUCING DEATH OF A CANCER CELL

(71) Applicant: Chung Shan Medical University, Taichung City (TW)

(72) Inventors: Chau-Jong Wang, Taichung City (TW); Mon-Yuan Yang, Taichung City (TW); Tzu-Hsin Chen, Taichung City (TW)

(73) Assignee: Chung Shan Medical University, Taichung City (TW)

(21) Appl. No.: 14/656,874

(22) Filed: Mar. 13, 2015

(51) Int. Cl. A61K 31/05; A61K 36/185

(52) U.S. Cl. CPC A61K 31/05; A61K 36/185

(57) ABSTRACT

The present invention provides a method for inducing death of a cancer cell in a subject in need thereof comprising administering to the subject a pharmaceutically effective amount of Nelumbo Nucifera leave water extract, wherein the Nelumbo Nucifera leave water extract comprises polyphenols.

### FIELD OF THE INVENTION

The present invention is related to a method for inducing death of a cancer cell by using Nelumbo Nucifera leave water extract.

### BACKGROUND OF THE INVENTION

Liver cancer or hepatic cancer is a cancer that originates in the liver. The leading cause of liver cancer is cirrhosis due to hepatitis B, hepatitis C, and alcohol. Liver cancers are formed from either the liver itself or from structures within the liver, including blood vessels or the bile duct. Primary liver cancer is the sixth most frequent cancer globally and the second leading cause of cancer death. In 2012 it occurred in 782,000 people and resulted in 746,000 deaths. Higher rates of liver cancer occur where hepatitis B and C are common, including East-Asia and sub-Saharan Africa. Five year survival rates are 17% in the United States.

The most frequent liver cancer, accounting for approximately 75% of all primary liver cancers, is hepatocellular carcinoma (HCC). HCC is a cancer formed by liver cells, known as hepatocytes, which become malignant. Most cases of HCC are secondary to either a viral hepatitis infection (hepatitis B or C) or cirrhosis (alcoholism being the most common cause of liver cirrhosis).

Hepatocellular carcinoma, like any other cancer, develops when there is a mutation to the cellular machinery that causes the cell to replicate at a higher rate and/or results in the cell avoiding apoptosis. In particular, chronic infections of hepatitis B and/or C can aid the development of hepatocellular carcinoma by repeatedly causing the body's own immune system to attack the liver cells, some of which are infected by the virus, others merely bystanders. While this constant cycle of damage followed by repair can lead to mistakes during repair which in turn lead to carcinogenesis, this hypothesis is more applicable, at present, to hepatitis C. Chronic hepatitis C causes HCC through the stage of cirrhosis. In chronic hepatitis B, however, the integration of the viral genome into infected cells can directly induce a non-cirrhotic liver to develop HCC. Alternatively, repeated consumption of large amounts of ethanol can have a similar effect. Besides, cirrhosis is commonly caused by alcoholism, chronic hepatitis B and chronic hepatitis C.

Sacred water lotus (Nelumbo nucifera) has been used in the Orient as a medicinal herb for well over 1,500 years. All parts of the plant are used as herbal medicine because they have astringent, cardio-tonic, febrifuge, hypotensive, resolvent, stomachic, styptic, tonic or vessel dilating effect. For example, the leaf juice is used in the treatment of diarrhoea and is decocted with liquorice for the treatment of sunstroke.

A decoction of the flowers is used in the treatment of premature ejaculation. The flowers are recommended as a cardiac tonic. A decoction of the floral receptacle is used in the treatment of abdominal cramps, bloody discharges etc. The flower stalk is haemostatic. It is used in treating bleeding gastric ulcers, excessive menstruation, post-partum haemorrhage.

The stamens are used in treating urinary frequency, premature ejaculation, haemolysis, epistasis and uterine bleeding. Besides, a decoction of the fruit is used in the treatment of agitation, fever, heart complaints etc. Furthermore, the seed is sedative and used in the treatment of poor digestion, enteritis, chronic diarrhoea, insomnia and palpitations.

The root is tonic and the root starch is used in the treatment of diarrhea or dysentery, a paste is applied to ringworm and other skin ailments. It is also taken internally in the treatment of haemorrhages, excessive menstruation and nosebleeds. The roots are harvested in autumn or winter and dried for later use.

The root nodes are used in the treatment of nasal bleeding, haemoptysis, haematuria and functional bleeding of the uterus. The plumule and radicle are used to treat thirst in high febrile disease, hypertension, insomnia and restlessness.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows The HPLC chromatogram of Nelumbo Nucifera leave water extract.

FIG. 2 shows the effect of Nelumbo Nucifera leave water extract supplement on body weight in the AAF-induced rats.

FIG. 3 shows that Nelumbo Nucifera leave water extract supplement ameliorated the liver appearance in AAF-induced HCC.

FIG. 4 shows that Nelumbo Nucifera leave water extract supplement ameliorated the biomarkers expression of hepatic injury and hepatocellular carcinoma.

FIG. 5 shows that Nelumbo Nucifera leave water extract supplement improved the formation of AAF-induced HCC.

FIG. 6 shows that Nelumbo Nucifera leave water extract supplement reduced lipid peroxidation in liver in AAF-induced HCC.

FIG. 7 shows that Nelumbo Nucifera leave water extract supplement enhanced activation of antioxidant enzymes in liver in AAF-induced HCC.

FIG. 8 shows that Nelumbo Nucifera leave water extract supplement enhanced the activation of GSTs in liver in AAF-induced HCC.

FIG. 9 shows that Nelumbo Nucifera leave water extract induced the cytotoxicity effects on Huh7 cell.

FIG. 10 shows that Nelumbo Nucifera leave water extract induced cell death in Huh7 cell.

FIG. 11 shows that Nelumbo Nucifera leave water extract induced cell apoptotic markers in Huh7 cell.

FIG. 12 shows that Nelumbo Nucifera leave water extract induced cellular ROS accumulation in a dose-dependent manner in Huh7 cell.

FIG. 13 shows that Nelumbo Nucifera leave water extract induced cellular ROS accumulation in a time-dependent manner in Huh7 cell.

FIG. 14 shows that Nelumbo Nucifera leave water extract induced mitochondrial dysfunction in a dose-dependent manner in Huh7 cell.

FIG. 15 shows that Nelumbo Nucifera leave water extract induced mitochondrial dysfunction in a time-dependent manner in Huh7 cell.

FIG. 16 shows that Nelumbo Nucifera leave water extract induced cytochrome c release from mitochondria.

FIG. 17 shows that Nelumbo Nucifera leave water extract induced the cytotoxicity effects on Hep3B cell.

FIG. 18 shows that Nelumbo Nucifera leave water extract induced cell death in Hep3B cell.

FIG. 19 shows that Nelumbo Nucifera leave water extract induced cell apoptotic markers in Hep3B cell.

FIG. 20 shows that Nelumbo Nucifera leave water extract induced autophagy in human hepatocellular carcinoma cell line.

FIG. 21 shows that Nelumbo Nucifera leave water extract induced acidic vesicular organelles (AVO) in human hepatocellular carcinoma cell line.

FIG. 22 shows that Nelumbo Nucifera leave water extract induced acidic vesicular organelles (AVO) formation in human hepatocellular carcinoma cell.

FIG. 23 shows that Nelumbo Nucifera leave water extract induced the relative protein expression of autophagy in Hep3B.

FIG. 24 shows that Nelumbo Nucifera leave water extract induced autophagic cell death in human hepatocellular carcinoma cell line.

### SUMMARY OF THE INVENTION

The present invention provides a method for inducing death of a cancer cell in a subject in need thereof comprising administering to the subject a pharmaceutically effective amount of Nelumbo Nucifera leave water extract, wherein the Nelumbo Nucifera leave water extract comprises polyphenols.

### DETAIL DESCRIPTION OF THE INVENTION

### Definitions

UNLWss otherwise specified, “a” or “an” means “one or more”.

The present invention provides a method for inducing death of a cancer cell in a subject in need thereof comprising administering to the subject a pharmaceutically effective amount of Nelumbo Nucifera leave water extract, wherein the Nelumbo Nucifera leave water extract comprises polyphenols.

According to the invention, the cancer cell is a hepatic cancer cell.

In one embodiment of the present invention, the pharmaceutically effective amount is 0.03 g/Kg to 1.5 g/Kg. In a more preferred embodiment of the present invention, the pharmaceutically effective amount is 0.06 g/Kg to 0.75 g/Kg. In another more preferred embodiment of the present invention, the pharmaceutically effective amount is 0.125 g/Kg to 0.5 g/Kg. In another more preferred embodiment of the present invention, the pharmaceutically effective amount is 0.04 g/Kg to 0.16 g/Kg.

According to the invention, the inducing death of the cancer cell is through inducing programmed cell death of the cancer cell and the programmed cell death is apoptosis or autophagy.

According to the invention, the administering is orally administering for a period of time from 1 month to 6 months.

The present invention also provides a method for treating or preventing cancer in a subject in need thereof comprising administering to the subject a pharmaceutically effective amount of Nelumbo Nucifera leave water extract, wherein the Nelumbo Nucifera leave water extract comprises polyphenols.

### EXAMPLES

The examples below are non-limiting and are merely representative of various aspects and features of the present invention.

### Preparation of Nelumbo Nucifera Leave Water Extract

The leaves of N. nucifera Gaertn. were purchased from Paiho Farmers' Association Organization in Tainan County, Taiwan. The fresh leaves were lyophilized and processed into a powdery form. The dried powder (200 g) was mixed with 5000 mL of ultrapure water and stirred magnetically for 1 hr, placed in the cold room overnight and vacuum filtrated with filter. The extract solution was vacuum concentrated at 40° C. and was dehydrated with vacuum freezing drying to obtain Nelumbo Nucifera leave water extract (NLW). When using, the lyophilized powder was weighed and prepared for different concentrations. The Nelumbo Nucifera leave water extract used for cell culture needs to be filtered through 0.22 μm filter first.

### Animals and Treatment

Male Wistar rats, age 4-5 weeks and weighing 140-160 g, were purchased from BioLASCO Taiwan Co., Ltd., kept at constant temperature at 22-24° C., and illuminated for 12 hrs daily (lights on from 06:00 to 18:00). All procedures involving laboratory animal experiment were in accordance with the guidelines of the Instituted Animal Care and Use Committee of Chung Shan Medical University (IACUC, CSMU) for the care and the use of laboratory animals. After 1 week maintenance for adaptation to the environment, the rats were randomly grouped by body weight. Normal diet (Laboratory Rodent Diet 5001) was purchased from PMI Nutrition International, which consisted of 23.0% crude protein, 4.5% crude fat, 6.0% crude fiber, and 8.0% ash as described in the manufacturer's instructions.

For AAF (2-acetylaminofluorene)-induced hepatic damage, the rats were divided into five groups, and each group contained 10 rats, which were fed on a unique diet for 6 months and weighed monthly. Diets for the five groups were (1) normal control, fed with the normal diet; (2) AAF, fed with the normal diet containing 0.03% AAF (w/w); (3) AAF+0.125 g/kg NLW, fed with the diet of (2) containing 0.125 g/kg NLW; (4) AAF+0.25 g/kg NLW, fed with the diet of (2) containing 0.25 g/kg NLW; and (5) AAF+0.5 g/kg NLW, fed with the diet of (2) containing 0.5 g/kg NLW. After 6 months of application of different diets, the blood and the whole liver were collected from rats that had been fasted 12-14 hrs and then were sacrificed. The whole livers were photographed, weighed, and then homogenized for protein extraction.

### Blood Parameters Analysis and Determination of TG and TC in the Liver

Plasma levels were measured on a Beckman Synchron CX9 clinical chemistry analyzer. Reagent sets for AST, ALT, ALP, TG, TC, HDL-C (high-density lipoprotein cholesterol) and LDL-C (low-density lipoprotein cholesterol) were provided by Beckman Coulter Co. Liver (0.2 g) was homogenized with chloroform-methanol (1:2, v/v). Liver lipids were extracted and processed.

### Measurement of Lipid Peroxidation and Antioxidant Enzymes

Lipid peroxidation was determined from the formation of malondialdehyde (MDA) based on the amount of thiobarbituric acid-reactive substances (TBARS). The procedure used for determining GSH with o-phtalaldehyde (OPA) was performed as previously described (Anal. Biochem., 2000, 280, 80-86). GSH-Px activity was determined spectrophotometrically according to the method of Lawrence and Burk published in 1976. The SOD assay was conducted using a modified Marklund and Marklund method described in 1974. Catalase activity was measured based on the ability of the enzyme to break down H₂O₂ according to a modification of the method proposed by Aebi.

### Protein Extraction

Liver samples (0.1 g) were homogenized in 1 mL of ice-cold lysis buffer (10 mM HEPES, pH 7.2, 10 mM KCl, 2 mM MgCl₂, 1 mM dithiothreitol, 0.1 mM EDTA, and 1 mM PMSF). Eighty microliters of 10% (v/v) Igepal CA-630 was added into the homogenate and gently mixed well. After 10 min of incubation on ice, the homogenate was centrifuged at 14000×g for 5 min, and then, the supernatant (cytosolic fraction) was transferred into a new 1.5 mL eppendorf and stored at −70° C. The pellet was resuspended with the ice-cold lysis buffer containing 0.8% (v/v) Igepal CA-630 and incubated on ice for 10 min. After centrifugation, the pellet was resuspended with nuclear extraction buffer (50 mM HEPES, pH7.2, 50 m MKCl, 300 mM NaCl, 1 mM dithiothreitol, 0.1 mM EDTA, 1 mM PMSF, and 20% v/v glycerol) and incubated on ice for 10 min. After centrifuging at 14000×g for 5 min, the supernatant (nuclear fraction) was transferred into a new 1.5 mL eppendorf and stored at −70° C. for analysis within 2 weeks.

### Immunoblot Analysis

After feeding on indicated diets for 6 months, livers were obtained and homogenized for protein extraction. The crude proteins of liver were separated in a 12.5% sodium dodecyl sulfate-polyacrylamide gel and transferred onto a nitrocellulose membrane as previously described (J. Agric. Food Chem. 2007, 55 (9), 3620-3628). The blot was subsequently incubated with 5% nonfat milk in phosphate-buffered saline (PBS) for 1 hr, probed with a primary antibody against GST-α, GST-μ, NOS2, Nrf2, GPx, SOD-1, catalase, or β-actin for 2 hrs, and then reacted with an appropriate peroxidase-conjugated secondary antibody for 1 hr. All incubations were carried out at 30° C., and intensive PBS washing was performed between each incubation. After the final PBS wash, the signal was developed by ECL chemiluminescence, and the relative photographic density was quantitated by image analysis system (Alpha Imager 2000, Alpha Innotech Corp., San Leandro, Calif.).

### Assay of GST Activity

The total GST activity and the activities for specific GST isoform were determined according to the method of Habig et al. (J. Biol. Chem. 1974, 249 (22), 7130-7139). using 4-chloro-7-nitrobenzofurazan (NBD-Cl) for GST-R and 1,2-dichloro-4-nitrobenzene (DCNB) for GST-μ. The enzyme activity was expressed as nanomoles of substrate-GSH conjugate produced per minute per milligram of cytosolic protein. The change in absorbance of GST-α and GST-μ was obtained at 419 and 345 nm, respectively, and the enzyme activity was calculated as nmol of NBD-Cl and DCNB conjugate formed min⁻¹ mg⁻¹ protein using a molar extinction coefficient of 9.6×10³ M⁻¹ cm⁻¹, respectively. The protein concentration was determined by Protein assay kit (Bio-Rad Laboratory, Watford, England) with bovine serum albumin as a standard.

### Histopathological Examination for Malignant Hepatoma

The livers were collected, cut into small pieces, fixed in 10% buffered neutral formalin, and embedded in paraffin as described (J. Ethnopharmacol. 1997, 56 (3), 193-200). Sections were cut at a thickness of 3-5 μm and stained with hematoxylin and eosin. The histopathological changes including cell morphology and cellular lipid vesicles were examined by light microscopy (400×).

### Cell Culture

The human liver cancer cell lines Hep3B and Huh7 were purchased from Bioresource Collection and Research Center, where cell lines had been tested free of Mycoplasma, bacteria, fungi, and cellular contamination. Hep3B Cell was maintained in minimum essential medium (Invitrogen Inc.) supplemented with 10% fetal bovine serum, 1.5 g/L sodium bicarbonate, 2 mM L-glutamine and 1 mM penicillin/streptomycin. Huh7 Cell was maintained in Dulbecco's Modified Eagle Medium with 10% fetal bovine serum, 2 mM L-glutamine and 1 mM penicillin/streptomycin. All cells were incubated in a humidified 5% CO₂ atmosphere at 37° C.

### Cell Viability

Cells were seeded in 24-well plates at a density of 5×10⁴/ml and treated with the indicated concentrations of Nelumbo Nucifera leave water extract for 24 hrs. After incubation, cytotoxicity was determined using an MTT assay. Briefly, MTT solution (0.5 mg/ml; Sigma-Aldrich, St. Louis, Mo., USA) was added to each well and incubated for 4 hrs at 37° C. After washing with phosphate buffered saline (PBS), the purple blue formazan was dissolved in 1 ml Isopropanol, and the absorbance was measured at 563 nm. Cell viability was proportional to the amount of formazan.

### Cell Cycle Analysis

The Hep3B cells (1×10⁷ cell/100 mm dish) were treated with different concentrations of Nelumbo Nucifera leave water extract for 24 hrs. At the end of the treatment, the cells were collected and fixed with ice-cold 70% ethanol overnight at −20° C. After centrifugation, the cell pellets were treated with 1 mL of PI solution (20 μg/mL PI, 20 μg/mL RNase A, 0.1% triton-X 100) at the room temperature for 15 min without light. Subsequently, the samples were analyzed in a FACS Calibur system (BD Biosciences, San Diego, Calif., USA) using CellQuest software to determine the percentages of the cell-cycle phases.

### Example 1

The Components of Nelumbo Nucifera leave water extract was shown in Table 1.

TABLE 1
Components of Nelumbo Nucifera leave water extract.

| | Nelumbo Nucifera leave water |
|---|---|
| | extract (%) |
| Polyphenol | |
| Gallic acid as STD | 19.34 ± 0.15 |
| Quercetin as STD | 13.67 ± 0.66 |
| Flavonoid | |
| Flavone & Flavonol | 1.63 ± 0.02 |
| Flavanone & Flavanonol | 4.26 ± 0.32 |
| Carbohydrate | 16.63 ± 1.22 |
| Protein | 4.55 ± 0.42 |
| Lipid | 2.43 ± 0.83 |

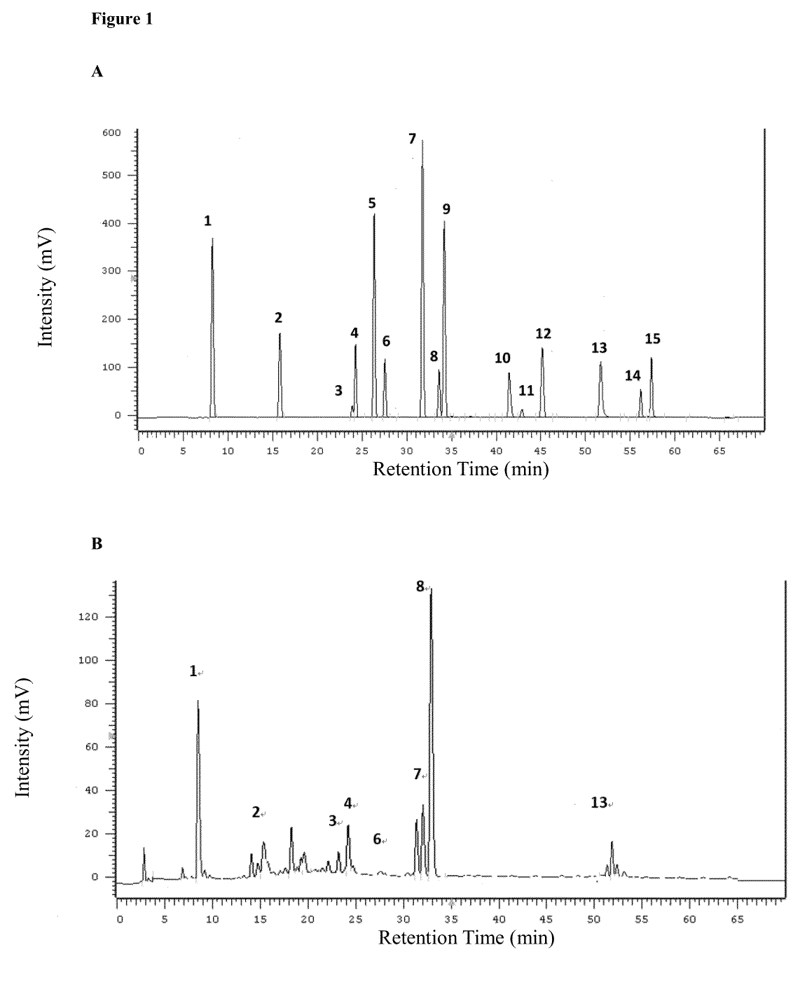

FIG. 1 showed The HPLC chromatogram of Nelumbo Nucifera leave water extract. HPLC chromatogram of fifteen kinds of standard polyphenols was shown in FIG. 1A. Peaks: 1, gallic acid (GA); 2, protocatechuic acid (PCA); 3, catechin (C); 4, gallocatechin gallate (GCG); 5, caffeic acid (CA); 6, epicatechin (EC); 7, p-coumaric acid (p-CA); 8, rutin (R); 9, ferulic acid (FA); 10, gossypin (G); 11, hesperetin (H); 12, resveratrol (RV); 13, quercetin (Q); 14, naringenin (N); 15, hydroxyflavin (FlOH). HPLC chromatogram of free polyphenols from Nelumbo Nucifera leave water extract was shown in FIG. 1B.

Composition of the Phenolic Compounds of Nelumbo Nucifera leave water extract by HPLC Analysis was shown in Table 2.

TABLE 2
Composition of the Phenolic Compounds of Nelumbo Nucifera leave
water extract by HPLC Analysis

| | | Retention time | |
|---|---|---|---|
| Peak no. | Assigned identity | (min) | % |
| 1 | GA | 8.34 | 22.35 ± 0.15 |
| 2 | PCA | 15.87 | 7.97 ± 0.23 |
| 3 | C | 23.49 | 2.38 ± 0.11 |
| 4 | GCG | 24.32 | 5.14 ± 0.31 |
| 6 | EC | 27.63 | 0.65 ± 0.07 |
| 7 | p-CA | 31.87 | 8.22 ± 0.19 |
| 8 | R | 33.71 | 36.53 ± 0.28 |
| 13 | Q | 51.97 | 4.03 ± 0.05 |

### Example 2

### The Change of Rat's Weight

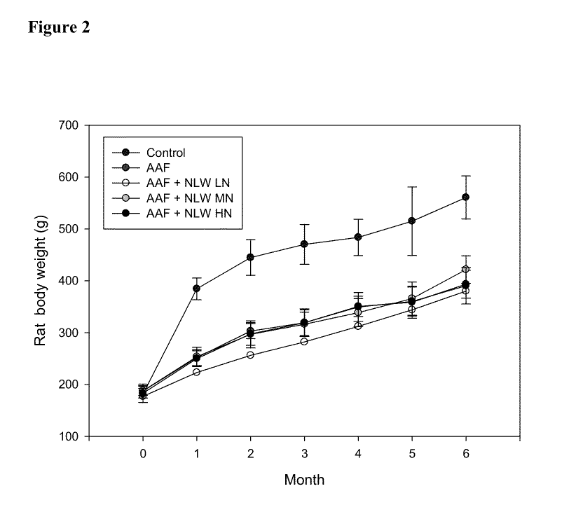

FIG. 2 showed the effect of Nelumbo Nucifera leave water extract supplement on body weight in the AAF-induced rats. Wistar rats fed on normal diet (control), normal diet containing AAF (AAF), normal diet containing AAF and Nelumbo Nucifera leave water extract 0.125 g/kg (AAF+NLW LN), normal diet containing AAF and Nelumbo Nucifera leave water extract 0.25 g/kg (AAF+NLW MN), and normal diet containing AAF and Nelumbo Nucifera leave water extract 0.5 g/kg (AAF+NLW FIN). The weights dropped significantly in AAF induced groups and showed that the growth and metabolism of the rats indeed changed with the AAF induction.

### The Influence of Nelumbo Nucifera Leave Water Extract on Liver Appearance and Weight

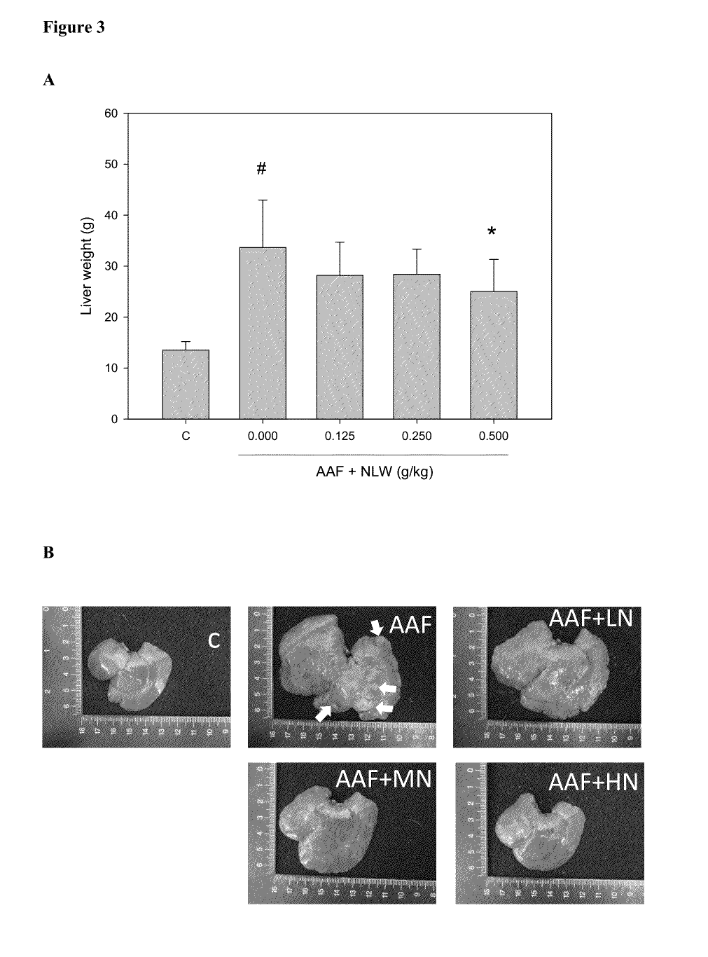

FIG. 3 showed that Nelumbo Nucifera leave water extract supplement ameliorated the liver appearance in AAF-induced HCC. The liver weight of the AAF-induced rats was shown in FIG. 3A. Wistar rats fed on C: normal diet; AAF: normal diet containing AAF; AAF+LN: normal diet containing AAF and NLW 0.125 g/kg; AAF+MN: normal diet containing AAF and NLW 0.25 g/kg; and AAF+FIN: normal diet containing AAF and NLW 0.5 g/kg. Data were shown as the means±SD; #, p<0.05 as compared with the control group; and *, p<0.05 as compared with the AAF group. FIG. 3B showed the effects of NLW on AAF-induced liver appearance of rats.

After sacrificing, the rat livers were weighted, recorded and analyzed. The liver weight/body weight was significantly higher in AAF treating group than in the normal group (FIG. 3A), which showed that the liver was damaged by the AAF stimulation and thus causing abnormal hypertrophy for about 3-fold. From the appearance of the rat liver, after AAF stimulation, it showed that there were many different sizes of abnormal nodules in liver which showed obvious phenomenon of liver tumors (FIG. 3B). For the group of AAF with 0.5 g/kg Nelumbo Nucifera leave water extract (NLW), the liver ratio and the occurrence of liver tumors had significantly reduced, showing the trend for retrieval of liver function and the ability of Nelumbo Nucifera leave water extract to protect the liver.

The influence of Nelumbo Nucifera leave water extract on the liver inflammation indicators of the blood

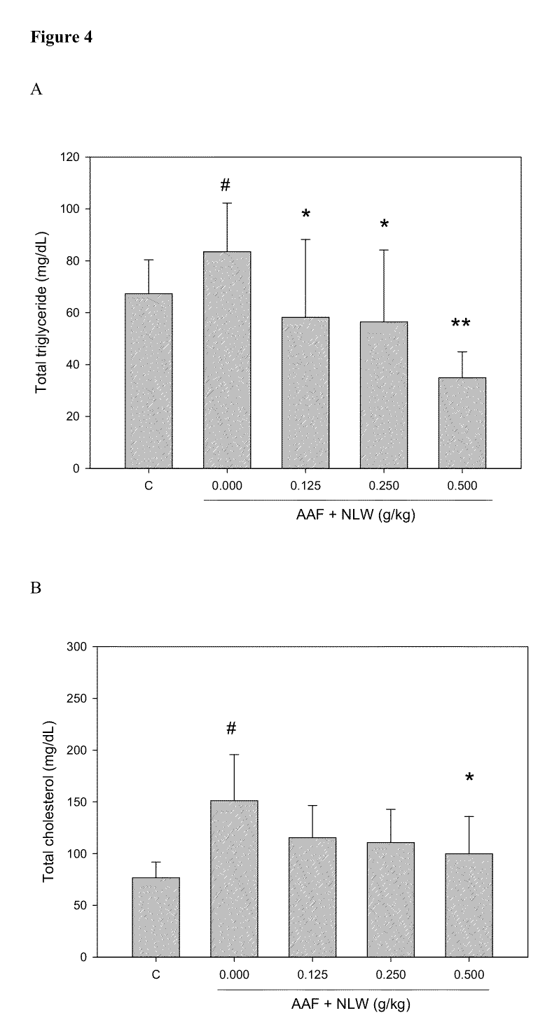

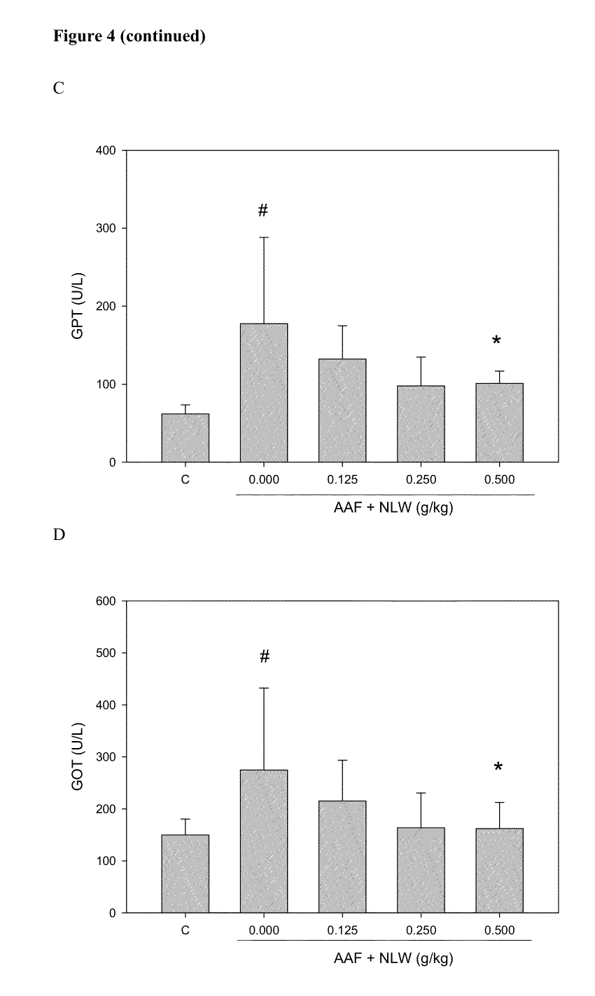

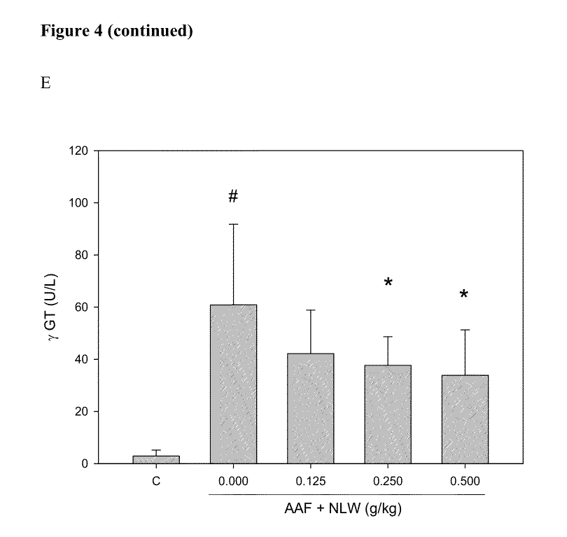

FIG. 4 showed that Nelumbo Nucifera leave water extract supplement ameliorated the biomarkers expression of hepatic injury and hepatocellular carcinoma. The biomarkers being quantitated were (A) total triglyceride, (B) total cholesterol, (C) GPT activity, (D) GOT activity, and (E) γ-GT level in plasma from AAF-induced rats. C: normal diet; AAF: normal diet containing AAF; 0.125: normal diet containing AAF and NLW 0.125 g/kg; 0.25: normal diet containing AAF and NLW 0.25 g/kg and 0.5: normal diet containing AAF and NLW 0.5 g/kg. Data were shown as the means±SD; #, p<0.05 as compared with the control group; *, p<0.05 as compared with the AAF group; and **, p<0.01 as compared with the AAF group.

The results showed that AAF indeed caused liver damage and the liver inflammation indicators GOT, GPT, γ-GT, total cholesterol and total triglyceride raised. As the feeding dose of the Nelumbo Nucifera leave water extract increased, the blood GOT, GPT, γ-GT, total cholesterol and total triglyceride values were effectively lowered to approaching the values of the normal group. Based on the above results, Nelumbo Nucifera leave water extract had the liver protecting ability to avoid liver damage (FIG. 4).

The Results of Liver Appearance and Histopathological Analysis

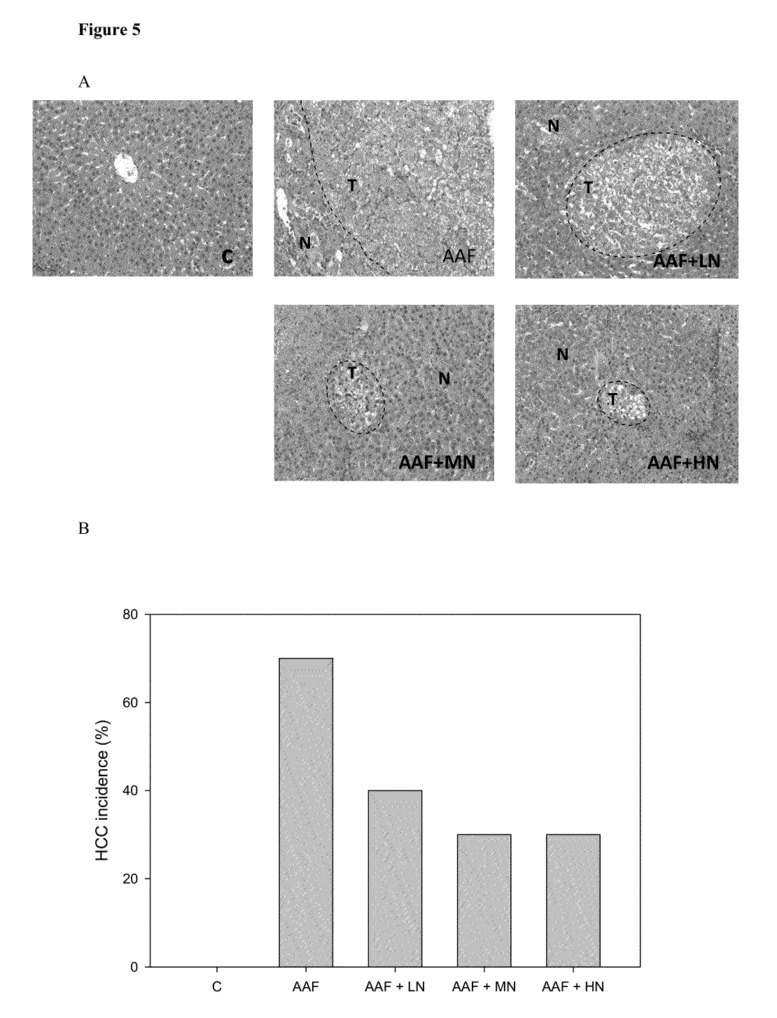

FIG. 5 showed that Nelumbo Nucifera leave water extract supplement improved the formation of AAF-induced HCC. The sections were stained with H&E and examined by microscope at 100× (FIG. 5A). N: normal hepatocyte; T: tumor. Livers from Wistar rats fed on C: normal diet; AAF: normal diet containing AAF; AAF+LN: normal diet containing AAF and NLW 0.125 g/kg; AAF+MN: normal diet containing AAF and NLW 0.25 g/kg; and AAF+HN: normal diet containing AAF and NLW 0.5 g/kg were fixed, embedded, and sectioned. FIG. 5B showed the incidence of Hepatocellular carcinoma in AAF-induced Rat.

H & E stain showed that liver cells arranged regularly and neatly in normal rat liver tissue and the hepatoma cells appeared nucleoli pleomorphism phenomenon with uneven sizes and the situation of hyperchromasia. AAF treated groups showed the occurrence of cancer cells and obvious vacuolization phenomenon. From the staining result of each group, with Nelumbo Nucifera leave water extract dose increased, the number of cancerous cells and the region had significantly reduced (FIG. 5A). With the increasing dose of Nelumbo Nucifera leave water extract, the occurrence rate of liver cancer in rats of each group slowed (FIG. 5B), and showed the gradually improvement of the liver.

### The Protecting Mechanism of Nelumbo Nucifera Leave Water Extract

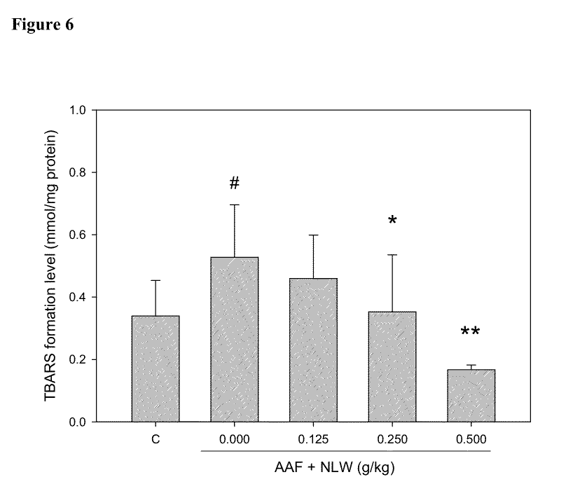

FIG. 6 showed that Nelumbo Nucifera leave water extract supplement reduced lipid peroxidation in liver in AAF-induced HCC. The levels of TBARS formation in liver was quantitated by spectrophotomic analysis. The crude lipid extracts of liver obtained from Wistar rats fed C: normal diet; 0.000: normal diet containing AAF; 0.125: normal diet containing AAF+NLW 0.125 g/kg; 0.25: normal diet containing AAF+NLW 0.25 g/kg; and 0.5: normal diet containing AAF+NLW 0.5 g/kg were investigated. Data were shown as the means±SD; #, p<0.05 as compared with the normal group; *, p<0.05 as compared with the AAF group, and **, p<0.01 as compared with the AAF group.

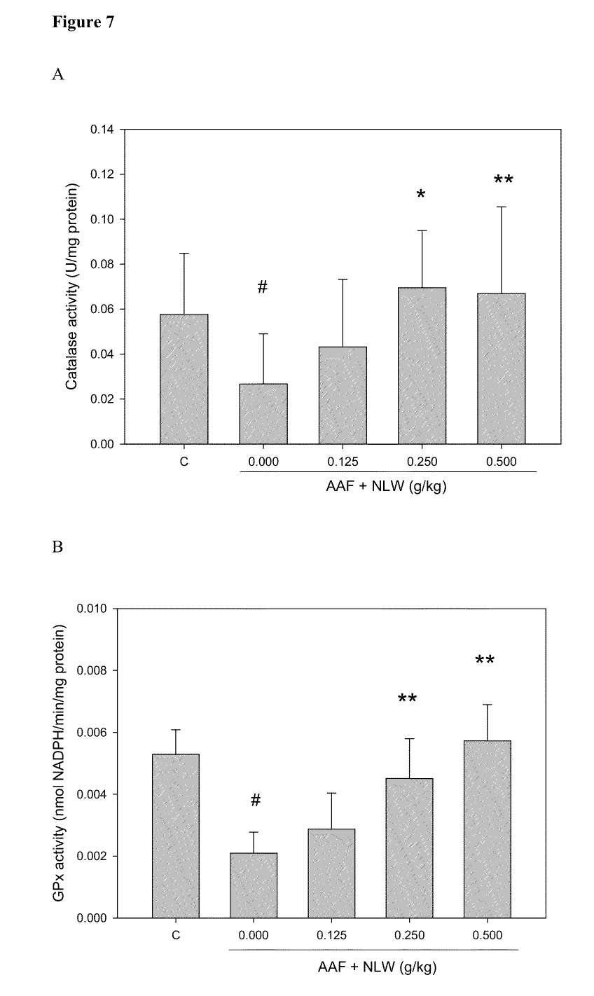

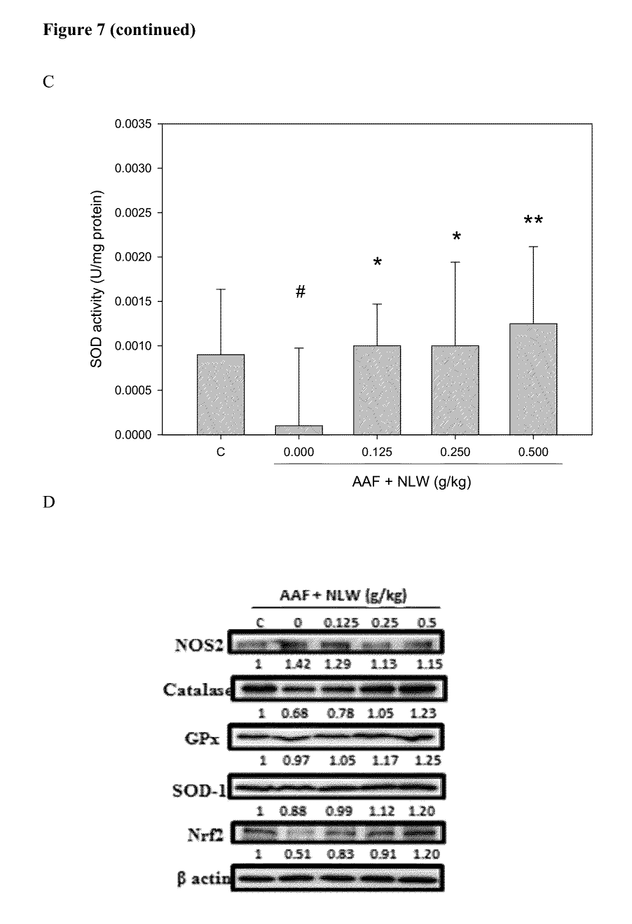

FIG. 7 showed that Nelumbo Nucifera leave water extract supplement enhanced activation of antioxidant enzymes in liver in AAF-induced HCC. (A) GPx, (B) SOD-1, and (C) catalase enzymatic activity in liver was quantitated by spectrophotomic analysis. The crude extracts of liver obtained from Wistar rats fed C: normal diet; 0.000: normal diet containing AAF; 0.125: normal diet containing AAF+NLW 0.125 g/kg; 0.25: normal diet containing AAF+NLW 0.25 g/kg; and 0.5: normal diet containing AAF+NLW 0.5 g/kg were investigated. Data were shown as the means±SDs; #, p<0.05 as compared with the normal group; *, p<0.05 as compared with the AAF group, and **, p<0.01 as compared with the AAF group. FIG. 7D showed the effects of Nelumbo Nucifera leave water extract on antioxidant enzymes expression in rats with hepatic fibrosis. The liver tissue extracts of control group, AAF group, AAF+NLW 0.125 g/kg group, AAF+NLW 0.25 g/kg group, and AAF+NLW 0.5 g/kg group, were subjected to Western blot to analysis NOS2, catalase, GPx, SOD-1, and Nrf2 expression. The levels of these proteins were subsequently quantitated by densitometric analysis with that control being 100%. Data were presented as mean±SD from three independent experiments.

TBARs assay showed that AAF induced large number of lipid peroxidation in liver and the situation improved with increasingly feeding dose of Nelumbo Nucifera leave water extract (FIG. 6). From the measurement of antioxidant enzymes catalase, GPx and superoxide dismutase (SOD) activity, the antioxidant enzyme activities of AAF groups were lower and with Nelumbo Nucifera leave water extract dose increased, the situation significantly improved (FIG. 7A-C). From the results of the western blot, Nelumbo Nucifera leave water extract increased the protein expression of antioxidant enzymes (FIG. 7D) and showed that Nelumbo Nucifera leave water extract restored the activity of antioxidant enzymes, providing liver protection.

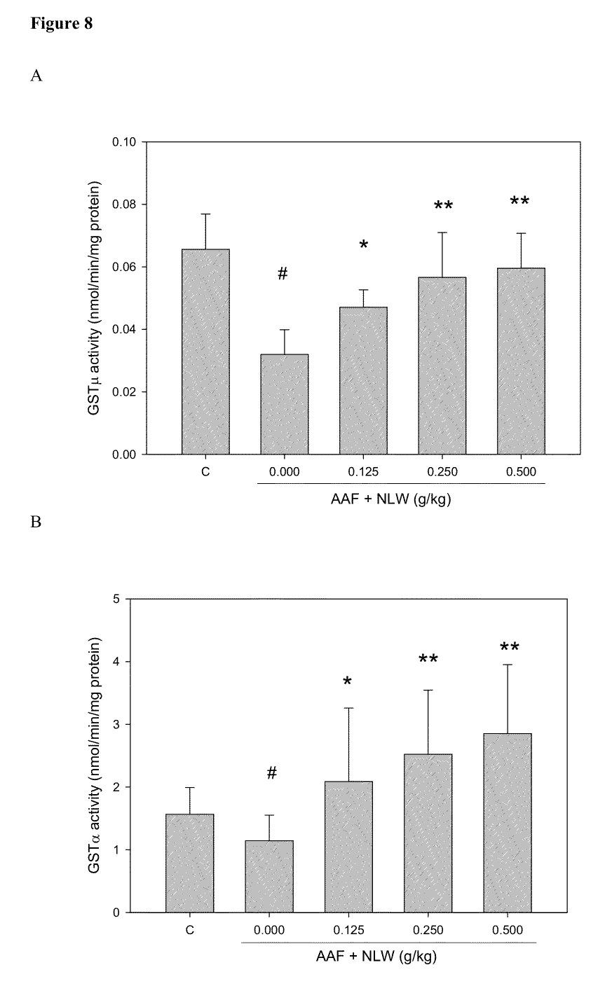

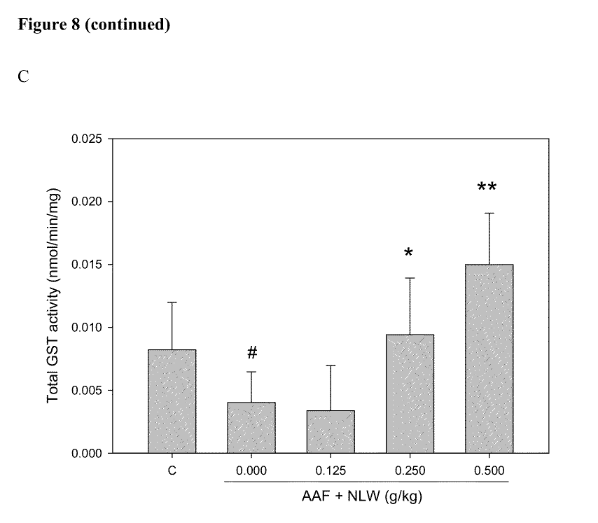

FIG. 8 showed that NLW supplement enhanced the activation of GSTs in liver in AAF-induced HCC. The crude extracts of liver obtained from Wistar rats fed on C: normal diet; 0,000: normal diet containing AAF; 0.125: normal diet containing AAF and NLW 0.125 g/kg; 0.25: normal diet containing AAF and NLW 0.25 g/kg; and 0.5: normal diet containing AAF and NLW 0.5 g/kg were investigated. The levels of (A) GST-μ (B) GST-α and (C) GST-total enzymatic activity were quantitated by spectrophotomic analysis. Data were shown as the means±SD; #, p<0.05 as compared with the normal group; *, p<0.05 as compared with the AAF group, and **, p<0.01 as compared with the AAF group.

The liver detoxification enzyme activity analysis showed that the enzyme activities of GST-α, GST-μ and total GST were lower in AAF inducing group, and with Nelumbo Nucifera leave water extract dose increased, the detoxification enzyme activity raised (FIG. 8A-C).

### Example 3

### Cytotoxicity Effects of Nelumbo Nucifera Leave Water Extract on Huh7 Cell

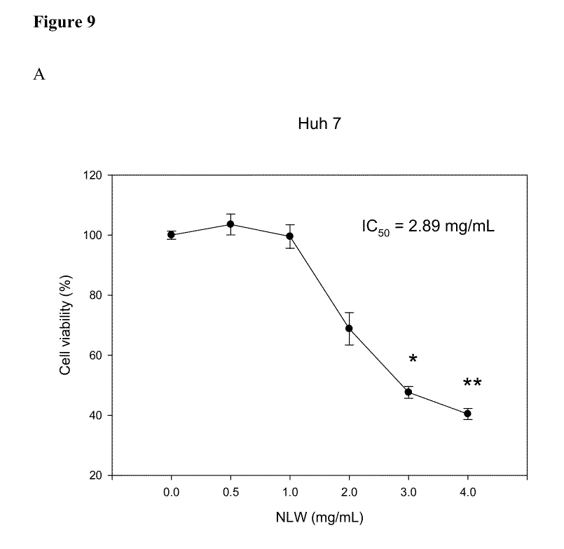

FIG. 9 showed that Nelumbo Nucifera leave water extract induced the cytotoxicity effects on Huh7 cell. Human hepatocellular carcinoma cell line, Huh7 cells, were incubated with different concentration Nelumbo Nucifera leave water extract (0.5-4 mg/mL) in 37° C. for 24 hrs. The data were means±SD for three replicates per treatment.

First, the effects of Nelumbo Nucifera leave water extract on Huh7 cells were determined using an MTT assay. Huh7 cells were exposed to different concentrations of Nelumbo Nucifera leave water extract (0, 0.25, 0.5, 1 and 2 mg/mL) under normal culture conditions. It showed that half lethal dose of Nelumbo Nucifera leave water extract for Huh7 was 2.89 mg/mL (FIG. 9).

### Nelumbo Nucifera Leave Water Extract Induced Cell Death of Huh7

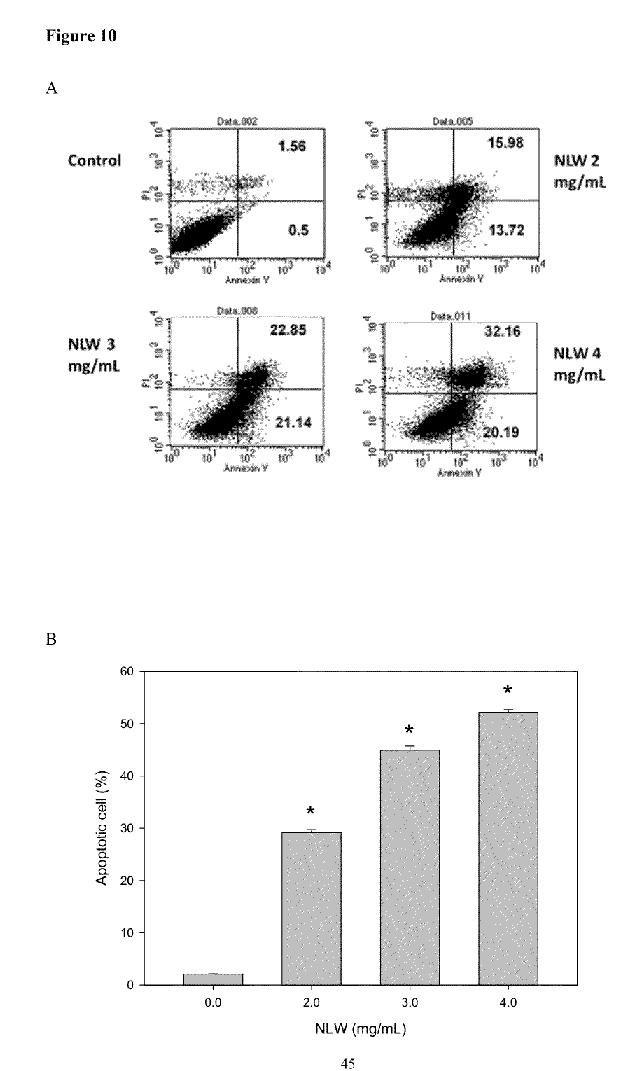

FIG. 10 showed that Nelumbo Nucifera leave water extract induced cell death in Huh7 cell. Apoptotic cell was evaluated after treating Huh7 cells with indicated concentration of Nelumbo Nucifera leave water extract. FIG. 10A showed that cell was stained with Annexin-V and PI. Flow cytometry profile represented Annexin-V-FITC staining in x axis and PI in y axis. FIG. 10B showed the quantitative assessment of the percentage of apoptosis cell. The data were means±SD for three replicates per treatment. *, p<0.05 as compared with control.

Flow cytometry was used to analyze the degree of hepatocellular carcinoma cell death caused by Nelumbo Nucifera leave water extract. The results from AnnexinV/PI double staining analysis showed that Nelumbo Nucifera leave water extract indeed caused Huh7 cell apoptosis, and with Nelumbo Nucifera leave water extract dose increased, the proportion of apoptotic cells also increased (FIG. 10).

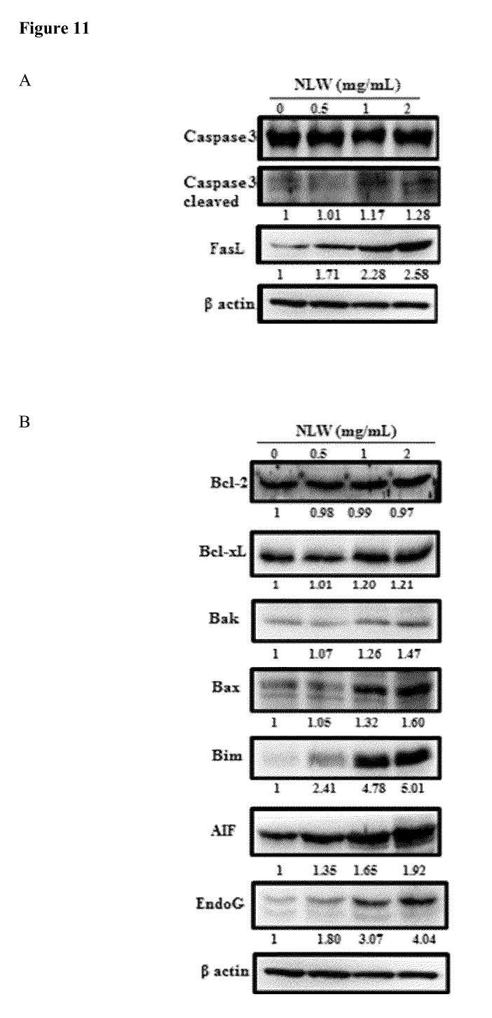

FIG. 11 showed that Nelumbo Nucifera leave water extract induced cell apoptotic markers in Huh7 cell. Immunoblot analysis showed that Caspase 3, FasL, Bcl₂ family, AIF, and EndoG protein expression of apoptosis in Huh7. Culture cells were treated with 0, 0.5, 1, and 2 mg/mL of Nelumbo Nucifera leave water extract for 12 hrs. Equal amount of total proteins were loaded in each lane of SDS-polyacrylamide gel (protein concentration was 50 μg/μL). Western hybridization was performed with antibodies against Caspase 3, FasL, Bcl₂ family, AIF, and EndoG. Western blot analysis of β-actin was used as an internal control.

It showed that the activation and protein expression of apoptosis-related protein Bcl2 family and caspase increased with the increasing dose of Nelumbo Nucifera leave water extract (FIG. 11). Therefore Nelumbo Nucifera leave water extract indeed caused apoptosis of human hepatoma cell line Huh7 and promoted cancer cell death.

### Nelumbo Nucifera Leave Water Extract Promoted Apoptosis Through Increasing Oxidative Stress of Huh7

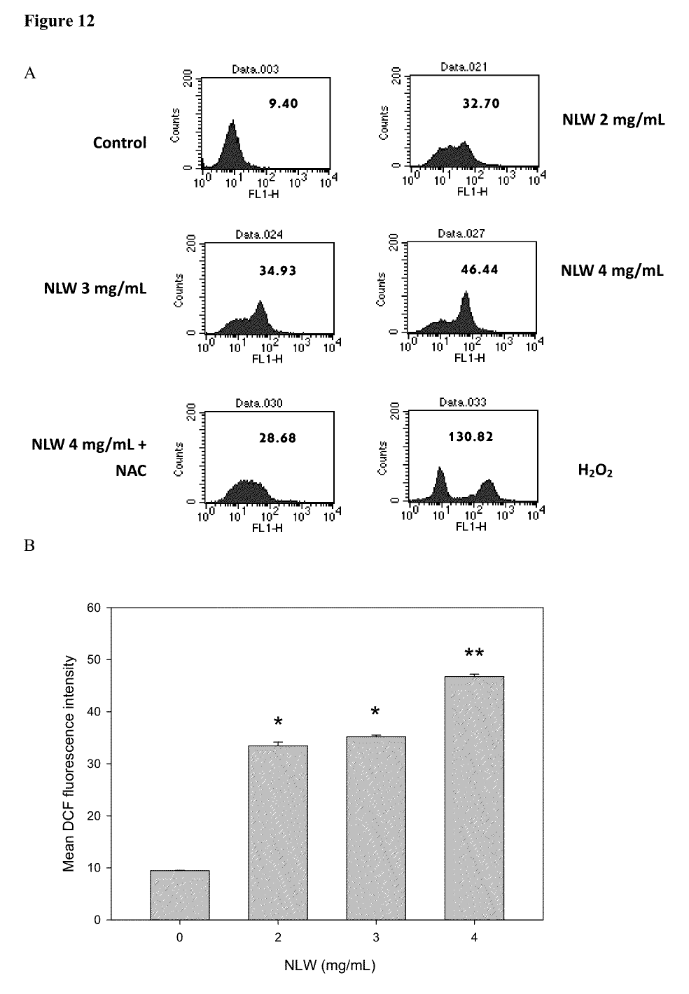

FIG. 12 showed that Nelumbo Nucifera leave water extract induced cellular ROS accumulation in a dose-dependent manner in Huh7 cell. FIG. 12A showed that Huh7 cells were treated with Nelumbo Nucifera leave water extract 2, 3, and 4 mg/mL or not, H₂O₂ as a positive control. NAC was an inhibitor for ROS. Measurement of intracellular ROS was detected by oxidized 2′,7′-dichlorofluorescein-diacetate (DCFH-DA) fluorescence. Data were analyzed by flow cytometry. Quantitative assessment of the mean of DCF fluorescence cell was shown in FIG. 12B. Significant differences versus control cells were represented by *, p<0.05 and **, p<0.01.

DCFH-DA (dye for ROS) was used to analyze intracellular ROS change after adding Nelumbo Nucifera leave water extract. The results from flow cytometry showed that after adding Nelumbo Nucifera leave water extract, intracellular ROS production raised (FIG. 12).

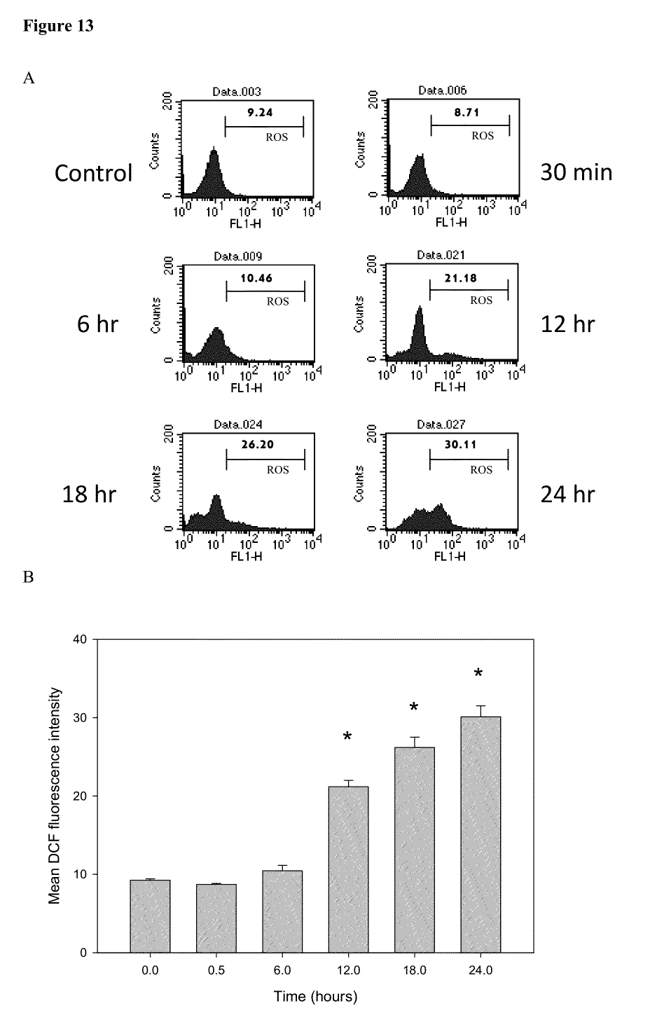

FIG. 13 showed that Nelumbo Nucifera leave water extract induced cellular ROS accumulation in a time-dependent manner in Huh7 cell. FIG. 13A showed that Huh7 cells were treated with NLW 2 mg/mL for different time periods (0.5, 6, 12, 18, and 24 hrs) or not. Measurement of intracellular ROS was detected by oxidized 2′,7′-dichlorofluorescein-diacetate (DCFH-DA) fluorescence. Data were analyzed for flow cytometry. FIG. 13B showed the quantitative assessment of the mean of DCF fluorescence cell.

The results showed that ROS in the cells reached the peak at about 12 hours, indicating the incense of ROS with time (FIG. 13). Therefore, Nelumbo Nucifera leave water extract indeed increased the production of excess ROS in Huh7 and caused the cell apoptosis with the tendency of dose and time dependent.

### Nelumbo Nucifera Leave Water Extract Promoted Apoptosis Through Inducing Mitochondrial Membrane Potential Loss of Huh7

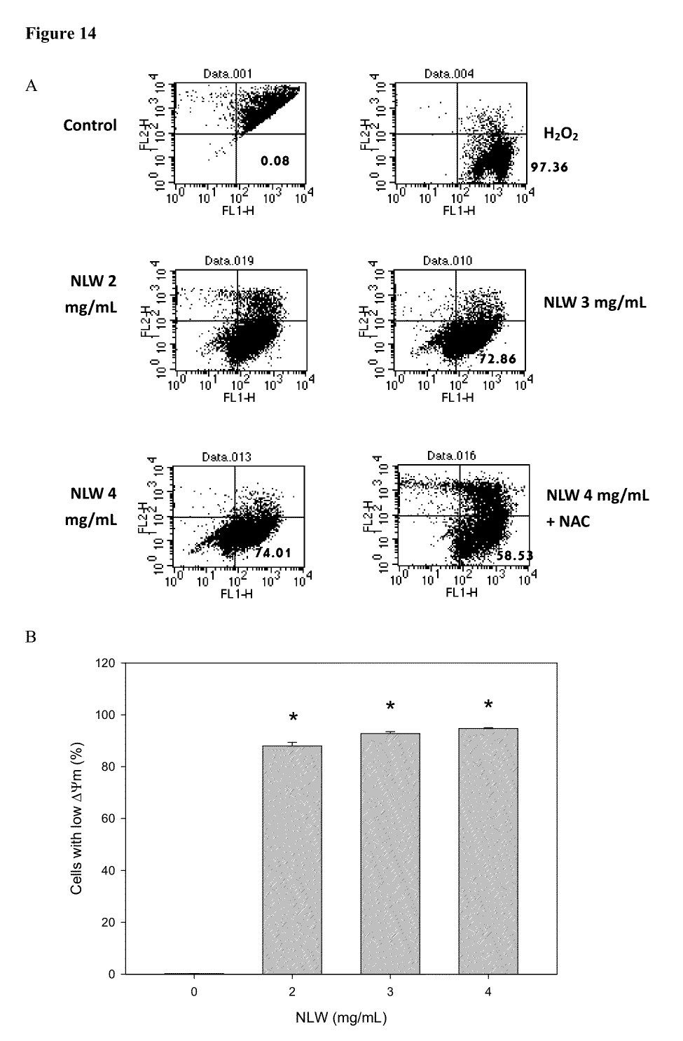

FIG. 14 showed that Nelumbo Nucifera leave water extract induced mitochondrial dysfunction in a dose-dependent manner in Huh7 cell. FIG. 14A showed the FACS analysis of Δψm in human hepatocellular carcinoma cell. Huh7 cell were treated with NLW 2, 3, and 4 mg/mL or not. H₂O₂ was used as a positive control. NAC was an inhibitor for ROS. Cells were stained with JC-1 and analyzed by flow cytometry. Quantitative assessment of the mean of Huh7 cells with low red and high green fluorescence which with mitochondrial depolarization were shown in FIG. 14 B.

JC-1 dye was used to measure the depolarization of the cell's mitochondria. When JC-1 entered into the mitochondria of healthy cells, it appeared red fluorescence, and when the mitochondrial membrane potential changed, JC-1 became green fluorescence. The results of flow cytometry analysis showed that mitochondrial membrane potential changed significantly after adding Nelumbo Nucifera leave water extract (FIG. 14).

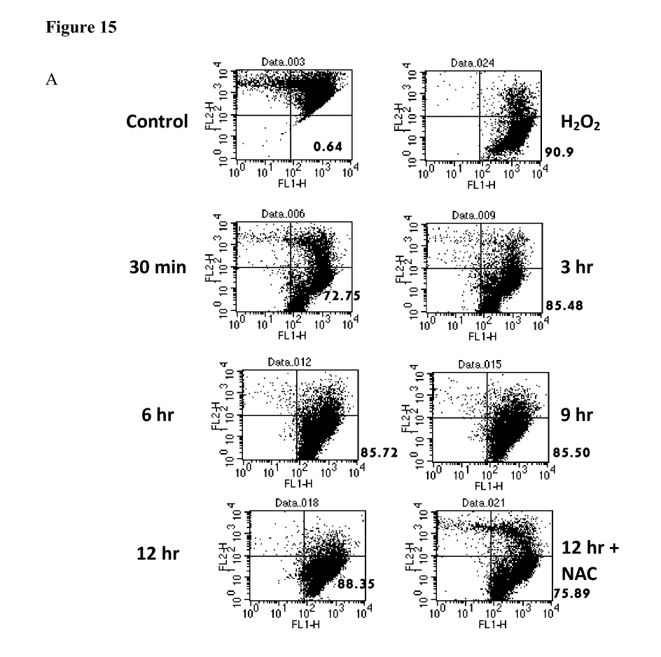

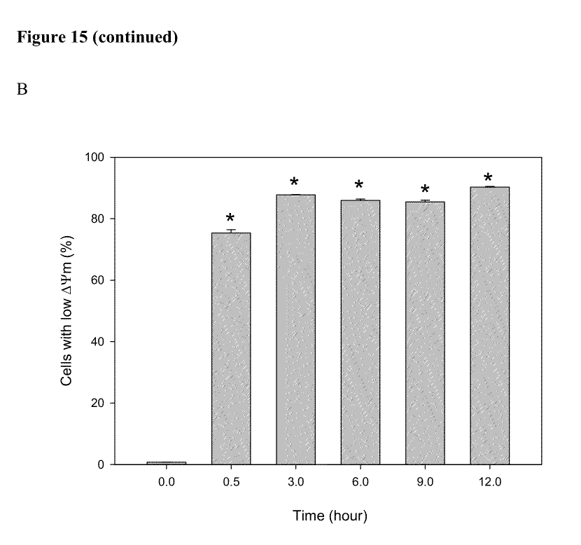

FIG. 15 showed that Nelumbo Nucifera leave water extract induced mitochondrial dysfunction in a time-dependent manner in Huh7 cell. FACS analysis of Δψm in human hepatocellular carcinoma cell was shown in FIG. 15A. Huh7 cells were treated with Nelumbo Nucifera leave water extract 2 mg/mL for different time periods (0.5, 3, 9, and 12 hrs) or not. H₂O₂ was used as a positive control. NAC was an inhibitor for ROS. Cells were stained with JC-1 and analyzed by flow cytometry. Photomultiplier settings were adjusted to detect JC-1 monomer fluorescence signals on the FL1 detector (green fluorescence) and JC-1 aggregate fluorescence signals on the FL2 detector (red fluorescence). Quantitative assessment of the mean of Huh7 cells with low red and high green fluorescence which with mitochondrial depolarization were shown in FIG. 15B.

30 minutes after adding Nelumbo Nucifera leave water extract, mitochondrial membrane potential changed significantly (FIG. 15). The results confirmed that Nelumbo Nucifera leave water extract prompted excess release of ROS through the change of intracellular mitochondrial membrane potential and causing cell apoptosis.

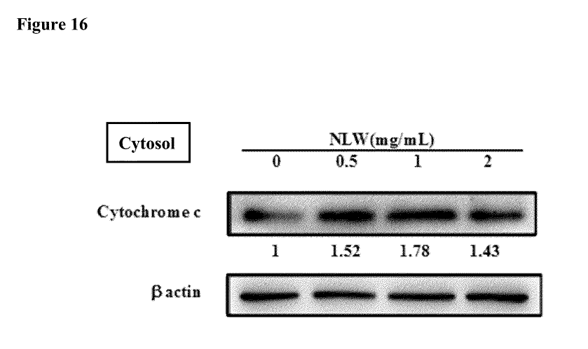

FIG. 16 showed that Nelumbo Nucifera leave water extract induced cytochrome c release from mitochondria. Immunoblot analysis of the cytochrome c in cytosolic subcellular fractions was shown. Equal amount of total proteins were loaded in each lane of SDS-polyacrylamide gel (protein concentration was 50 μg/μL). Western hybridization was performed with antibodies against cytochrome c. Western blot analysis of β-actin was used as an internal control.

The expression amounts of mitochondria and apoptosis associated protein Bcl-2 family were found significantly increased (FIG. 11). It was also found that cytochrome c released into the cytosol form mitochondria (FIG. 16). This confirmed that the Nelumbo Nucifera leave water extract did cause cell apoptosis through the regulation of mitochondria.

### Cytotoxicity Effects of Nelumbo Nucifera Leave Water Extract on Hep3B

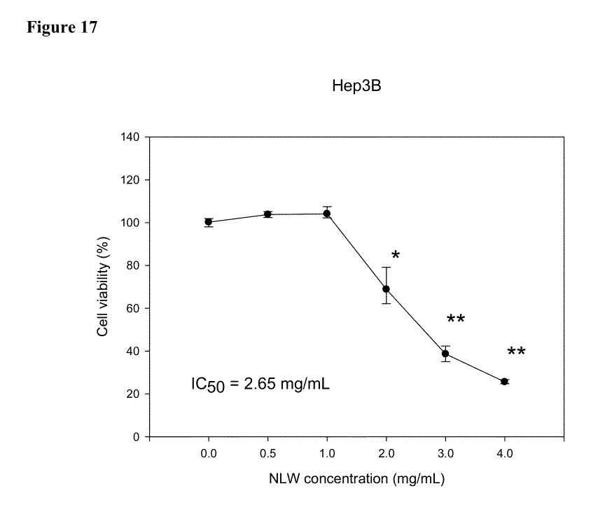

FIG. 17 showed that Nelumbo Nucifera leave water extract induced the cytotoxicity effects on Hep3B cell. Human hepatocellular carcinoma cell line, Hep3B cells, were incubated with different concentration Nelumbo Nucifera leave water extract (0.5-4 mg/mL) in 37° C. for 24 hrs. The data were means±SD for three replicates per treatment.

Cytotoxicity effect of Nelumbo Nucifera leave water extract was tested for another human hepatoma cell line Hep3B. Cells were treated with different doses of Nelumbo Nucifera leave water extract (0, 0.25, 0.5, 1 and 2 mg/mL) and processed MTT test. It showed that half lethal dose of Nelumbo Nucifera leave water extract for Hep3B was 2.65 mg/mL (FIG. 17).

### Nelumbo Nucifera Leave Water Extract Induced Hep3B Cell Apoptosis

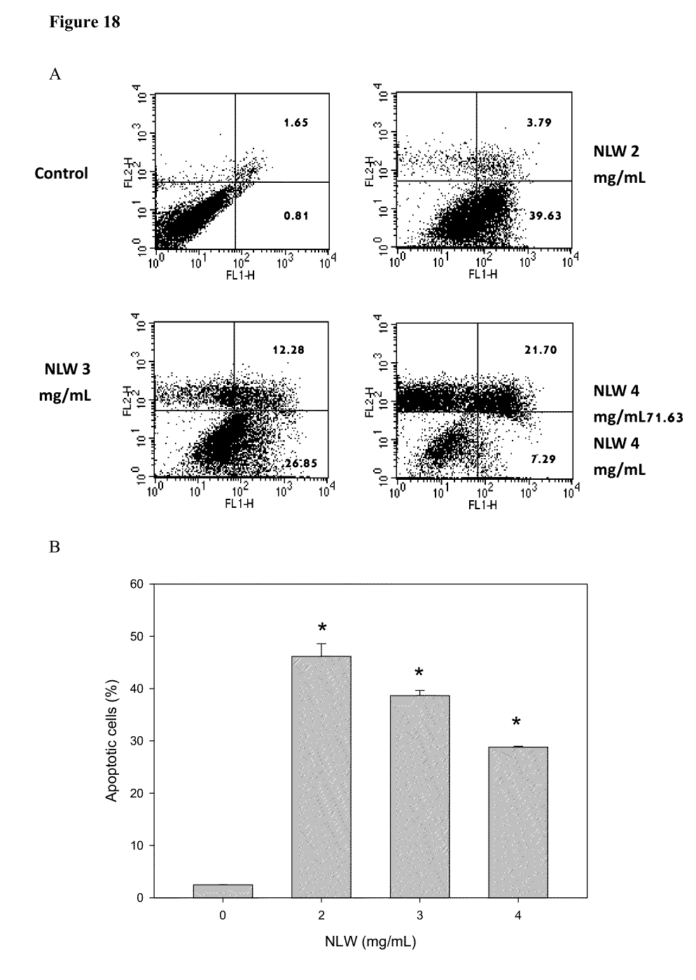

FIG. 18 showed that Nelumbo Nucifera leave water extract induced cell death in Hep3B cell. Apoptotic cell was evaluated after treating Hep3B cells with indicated concentration of Nelumbo Nucifera leave water extract. FIG. 18A showed that Cell was stained with Annexin-V and PI. Flow cytometry profile represented Annexin-V-FITC staining in x axis and PI in y axis. Quantitative assessment of the percentage of apoptosis cell was shown in FIG. 18B. The data were means±SD for three replicates per treatment. *, p<0.05 as compared with control.

The apoptosis extent of hepatocellular carcinoma caused by Nelumbo Nucifera leave water extract was tested by flow cytometry analysis. The AnnexinV/PI double staining analysis showed that Nelumbo Nucifera leave water extract indeed caused Hep3B apoptosis (FIG. 18).

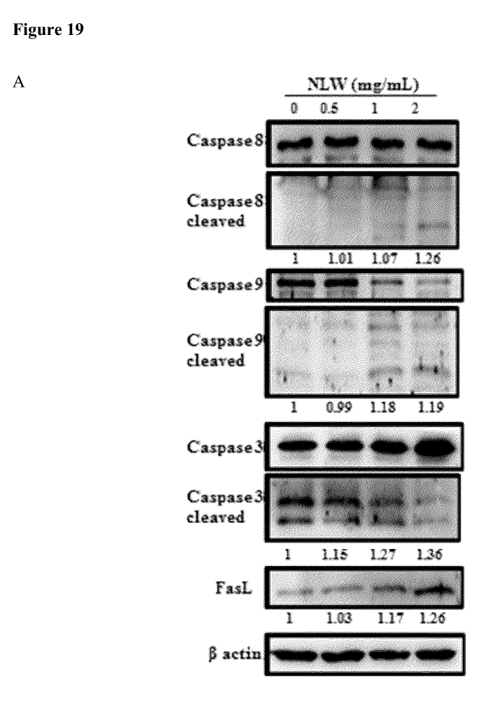

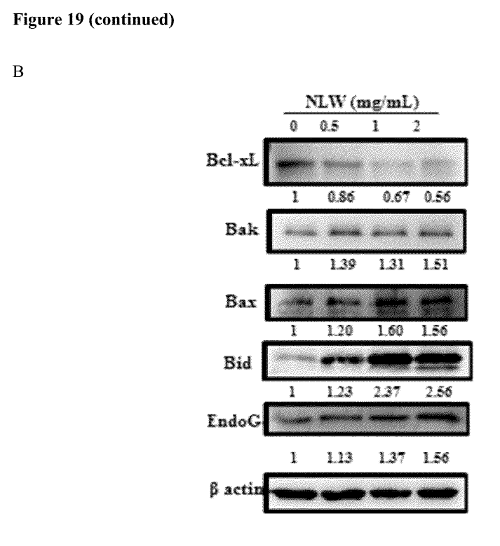

FIG. 19 showed that Nelumbo Nucifera leave water extract induced cell apoptotic markers in Hep3B cell. Immunoblot of the Caspase family, FasL, Bcl₂ family, and EndoG protein expression of apoptosis in Hep3B was analyzed. Culture cells were treated with 0, 0.5, 1, and 2 mg/mL of Nelumbo Nucifera leave water extract for 12 hrs. Equal amount of total proteins were loaded in each lane of SDS-polyacrylamide gel (protein concentration is 50 μg/μL). Western hybridization was performed with antibodies against Caspase family, FasL, Bcl₂ family, and EndoG. Western blot analysis of β-actin was used as an internal control. The data were means±SD for three replicates per treatment.

It showed that the activation and protein expression of apoptosis-related protein Bcl2 family and caspase increased with the increasing dose of Nelumbo Nucifera leave water extract (FIG. 19). Therefore Nelumbo Nucifera leave water extract indeed caused apoptosis of human hepatoma cell line Hep3B and promoted cancer cell death.

### Nelumbo Nucifera Leave Water Extract Induced Autophagy in Hep3B

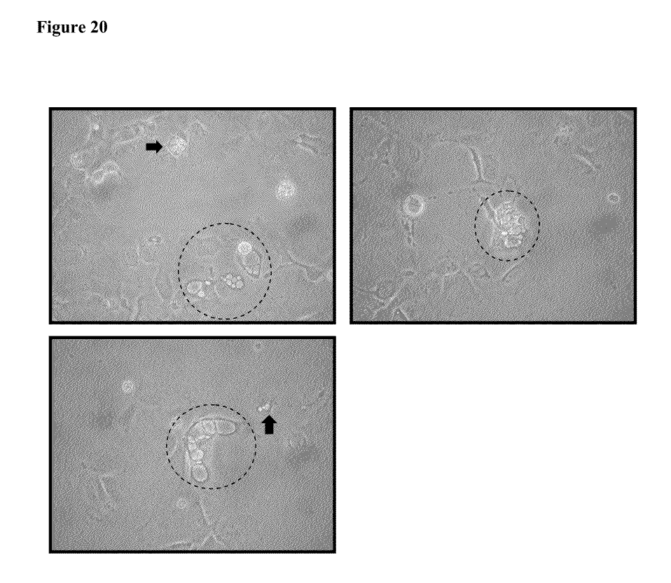

FIG. 20 showed that Nelumbo Nucifera leave water extract induced autophagy in human hepatocellular carcinoma cell line. Vesicular-like organelles (arrows and circle) from Nelumbo Nucifera leave water extract treated Hep3B cells which morphologies were observed and photographed by a reverse-phase microscope.

It was found that after treating with Nelumbo Nucifera leave water extract, Hep3B had different consequence from Huh7 that there were large number of vacuoles in Hep3B (FIG. 20).

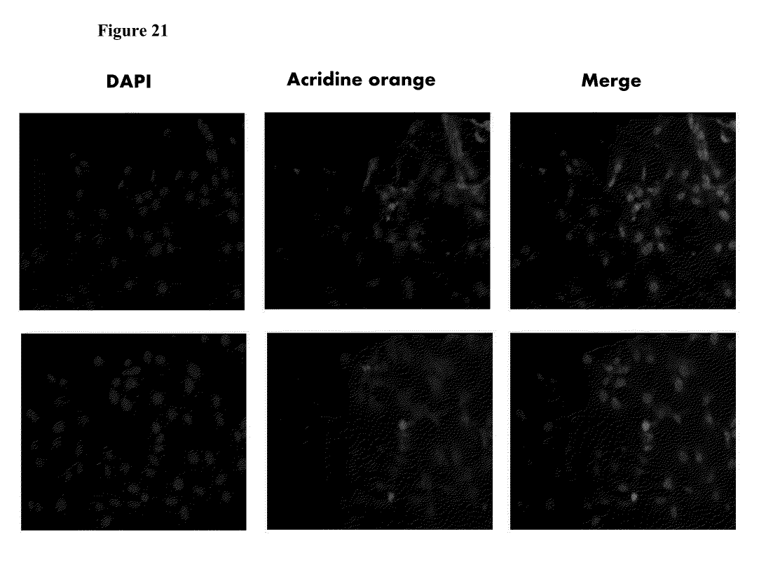

FIG. 21 showed that Nelumbo Nucifera leave water extract induced acidic vesicular organelles (AVO) in human hepatocellular carcinoma cell line. Microscopic image of Nelumbo Nucifera leave water extract treated Hep3B cells stained with 4′,6-diamidino-2-phenylindole (DAPI) (left panel), acridine orange (middle panel) and merge (right panel) were shown.

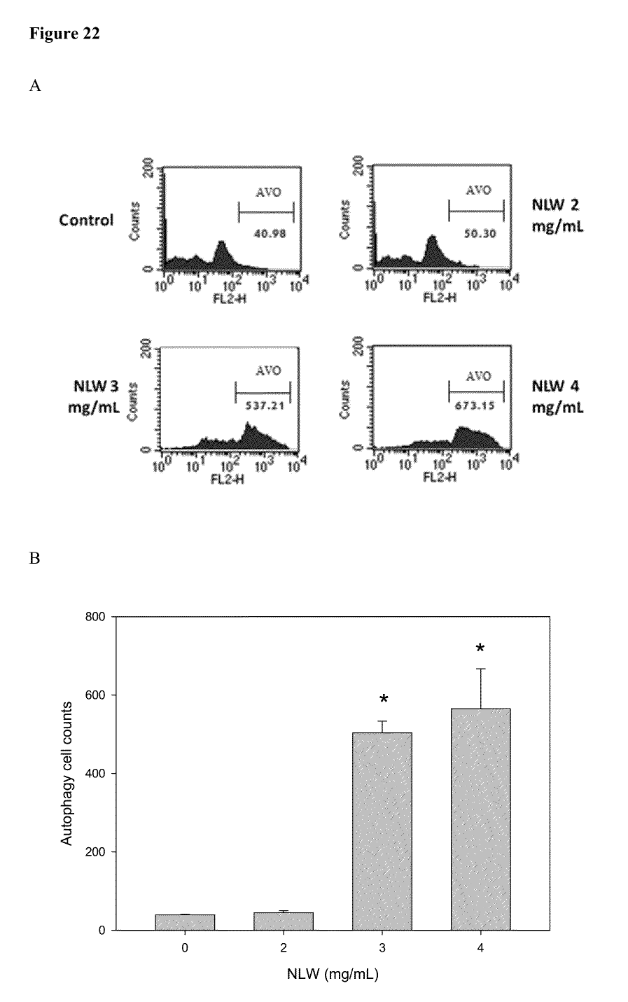

FIG. 22 showed that Nelumbo Nucifera leave water extract induced acidic vesicular organelles (AVO) formation in human hepatocellular carcinoma cell. FIG. 22A showed that Hep3B cells were treated with NLW 2, 3, and 4 mg/mL or not. Measurement of AVO was detected by acridine orange (AO) fluorescence. Data were analyzed by flow cytometry. Quantitative assessment of the mean of DCF fluorescence cell was shown in FIG. 22B. Significant differences versus control cells were represented by *, p<0.05. The data were means±SD for three replicates per treatment.

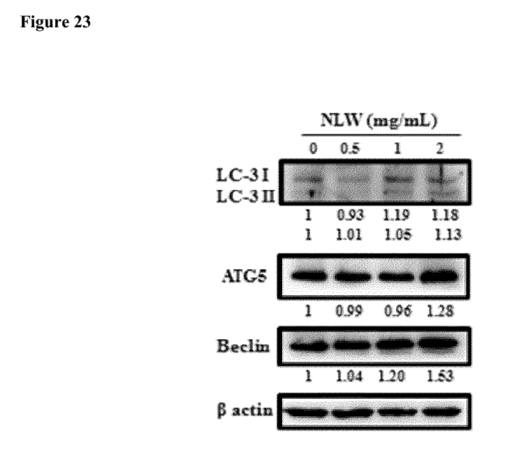

FIG. 23 showed that Nelumbo Nucifera leave water extract induced the relative protein expression of autophagy in Hep3B. Culture cells were treated with 0, 0.5, 1, and 2 mg/mL of NLW for 12 hrs. Equal amount of total proteins were loaded in each lane of SDS-polyacrylamide gel (Protein concentration is 50 μg/μL). Western hybridization was performed with antibodies against LC-3 I/II, ATG5, and Beclin1. Western blot analysis of β-actin was used as an internal control

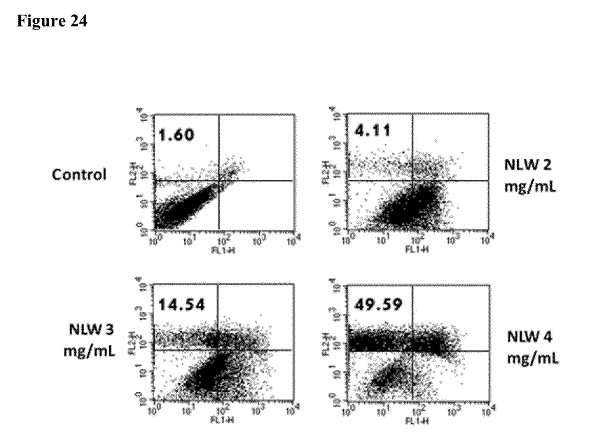

FIG. 24 showed that Nelumbo Nucifera leave water extract induced autophagic cell death in human hepatocellular carcinoma cell line. Autophagic death cell was evaluated after treating Hep3B cells with indicated concentration of Nelumbo Nucifera leave water extract. The percentage of autophagic death cells was calculated as the percentage of PI positive and Annexin-V negative cells. Cell was stained with Annexin-V and PI. Flow cytometry profile represented Annexin-V-FITC staining in x axis and PI in y axis. The data were means±SD for three replicates per treatment. Acidic vesicular organelles (AVO) formed when autophagy occurred. Acridine orange dye stained AVO and presented orange fluorescence which was observed by fluorescent microscope (FIG. 21) and quantitative analyzed by flow cytometry (FIG. 22). The results showed that large amount of acidic vesicular organelles were produced after adding Nelumbo Nucifera leave water extract. The expression amount of autophagy specific protein (LC-3, ATG5 and Beclin1) observed by western blot showed that Nelumbo Nucifera leave water extract indeed caused Hep3B cell autophagy (FIG. 23). In addition, the literature from the past confirmed that autophagy caused cell death. The results showed that Nelumbo Nucifera leave water extract indeed increased the number of cell death due to autophagy (FIG. 24).

Taken the above, Nelumbo Nucifera leave water extract induced apoptosis and autophagy of Hep3B cell and promoted the cell death. From the animal and cellar experiments, it showed that induced cell death was sufficient to elicit tumor regression following Nelumbo Nucifera leave water extract treatment to the subjects.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The apparatus, processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for inducing death of a cancer cell in a subject in need thereof comprising administering to the subject a pharmaceutically effective amount of Nelumbo Nucifera leave water extract, wherein the Nelumbo Nucifera leave water extract comprises polyphenols.

2. The method of claim 1, wherein the cancer cell is a hepatic cancer cell.

3. The method of claim 1, wherein the pharmaceutically effective amount is 0.03 g/Kg to 1.5 g/Kg.

4. The method of claim 1, wherein the pharmaceutically effective amount is 0.06 g/Kg to 0.75 g/Kg.

5. The method of claim 1, wherein the pharmaceutically effective amount is 0.125 g/Kg to 0.5 g/Kg.

6. The method of claim 1, wherein the pharmaceutically effective amount is 0.04 g/Kg to 0.16 g/Kg.

7. The method of claim 1, wherein the inducing death of the cancer cell is through inducing programmed cell death of the cancer cell.

8. The method of claim 7, wherein the programmed cell death is apoptosis or autophagy.

9. The method of claim 1, wherein the administering is orally administering.

10. The method of claim 1, wherein the administering is for a period of time from 1 month to 6 months.
